(19) United States
(12) Patent Application Publication
(10) Pub. No.: US 2010/0256687 A1
(43) Pub. Date: Oct. 7, 2010

(54) Fixation Device and Method of Use for a Ludloff Osteotomy Procedure

(75) Inventors: Steven Neufeld, Washington, DC (US); Albert Austin, Glen Burnie, MD (US); Emmanuel Anapliotis, Berlin (DE); Curt Kranz, Berlin (DE)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Merete Medical GmbH, Berlin (DE)

(21) Appl. No.: 12/726,195

(22) Filed: Mar. 17, 2010

Related U.S. Application Data
(60) Provisional application No. 61/165,680, filed on Apr. 1, 2009.

(51) Int. Cl. A61B 17/80

(52) U.S. Cl. 606/289; 606/280

(57) ABSTRACT

An internal plate fixation device is provided for load bearing and non-load bearing fixation during and after a Ludloff osteotomy procedure for hallux valgus correction in the first metatarsal bone of the foot. The fixation device includes a U-shaped plate having a straight section interconnecting a pair of opposed legs which are bent relative to the straight section. The straight section is formed therethrough with a set of non-threaded holes for receiving non-locking screws therein, and the legs are formed therethrough with a set of threaded holes for receiving locking screws therein. The straight section and the non-locking screws are configured to engage a dorsal portion of the first metatarsal bone. The legs and the locking screws are configured to engage a medial portion of the first metatarsal bone.

### CROSS REFERENCE TO RELATED APPLICATION

The present application relates to and claims priority from U.S. Provisional Patent Application No. 61/165,680, filed Apr. 1, 2009, which is fully incorporated herein by reference.

### FIELD

The present disclosure relates generally to foot and ankle surgery, and more particularly, pertains to a fixation device used in a Ludloff osteotomy procedure for surgically correcting a hallux valgus deformity in the first metatarsal bone of the foot.

### SUMMARY

The present inventors have recognized that the prior art does not disclose any internal fixation plate or method of use that is particularly useful in the Ludloff osteotomy, provides for rigid low profile and precise fixation, and permits non-load bearing or load bearing post operative care of a person having undergone surgical repair of the hallux valgus.

The present inventors thus sought to provide an internal fixation plate that allows for osteotomy surgical correction, initial placement of the internal fixation plate, completion and fixation of osteotomy correction and final internal fixation of the osteotomy.

In one example disclosed herein, an internal fixation device used in fixation of a metatarsal bone includes a U-shaped plate having a straight section interconnecting a pair of opposed legs which are bent relative to the straight section. The straight section is formed therethrough with a set of non-threaded holes, and the legs are formed therethrough with a set of threaded holes. Preferably, the straight section includes a pair of spaced apart non-threaded holes, and each leg has a single threaded hole. The non-threaded holes are located at opposite ends of the straight section, and the threaded holes are located at adjacent outermost edges of the legs. In a top view of the plate, the legs are bent rearwardly of the straight section with one of the legs having a length that is longer than the length of the other leg. The straight section has a thickness that is greater than the thickness of the legs to allow bending of the legs beyond an initial bent curvature thereof. The non-threaded holes define temporary and final fixation holes adapted to be used in fixation of the metatarsal bone. The threaded holes define final fixation holes adapted to be used in fixation of the metatarsal bone. The non-threaded holes are configured to receive non-locking screws therein, and the threaded holes are configured to receive locking screws therein. The threaded holes are differently sized to receive differently sized locking screws. One of the legs is formed with a temporary fixation hole adapted to be used in fixation of the metatarsal bone. The plate is configured to extend across an osteotomy of the metatarsal bone. The straight section is configured to engage a dorsal portion of the metatarsal bone, and the legs are configured to engage a medial portion of the metatarsal bone. The plate is configured to be engaged on the metatarsal bone during and after a Ludloff osteotomy.

In another example, an internal fixation device used in fixation of a metatarsal bone includes a U-shaped plate having a straight section interconnecting a pair of proximal and distal opposed legs which are bent relative to the straight section. The straight section is formed therethrough with a proximal non-threaded hole and a distal non-threaded hole, and each leg is formed therethrough with a threaded hole at a distal end of the each leg. A proximal non-locking bone fastener is received in the proximal non-threaded hole. A distal non-locking bone fastener is received in a distal non-threaded hole. A proximal locking bone fastener is received in the threaded hole of the proximal leg. A distal locking bone fastener is received in the threaded hole of the distal leg. Preferably, each bone fastener is a cannulated screw adapted to receive a K-wire. The non-locking bone fasteners include shafts which are partially threaded along lower portions thereof. The locking bone fasteners include shafts which are threaded along substantially entire lengths thereof. Walls forming the non-threaded holes are configured such that the bone fasteners received therein toggle to assume a plurality of angles relative to central axes of the non-threaded holes. The straight section and the non-locking bone fasteners are configured to engage a dorsal portion of the metatarsal bone. The plate and the bone fasteners are typically constructed of a titanium alloy Ti-6A1-4V.

In another example, a surgical method of fixating a Ludloff osteotomy of a first metatarsal bone to correct a hallux valgus deformity includes the steps of a) providing a U-shaped plate configured to be engaged on the first metatarsal bone across the Ludloff osteotomy, the plate having a straight section interconnecting a pair of proximal and distal opposed legs which are bent relative to the straight section, wherein the straight section is formed therethrough with a proximal non-threaded hole and a distal non-threaded hole, and wherein each leg is formed therethrough with a threaded hole at a distal end of each leg; b) entering the tissues of the foot affected with the hallux valgus deformity using a medial incision at a site on the first metatarsal bone to gain exposure thereto; c) from the medial side of the first metatarsal bone, performing a partial three-quarter Ludloff osteotomy cut at the site extending obliquely from dorsal-proximal to plantar-distal; d) positioning the plate across the partial osteotomy cut such that the straight section engages a dorsal portion of the first metatarsal bone and the legs engage a medial portion of the first metatarsal bone; e) temporarily fixating the plate at a proximal-dorsal location of the partial cut by inserting a proximal non-locking screw through the proximal non-threaded hole of the plate into the first metatarsal bone; f) completing the osteotomy cut forming dorsal and planar metatarsal fragments with the plate and the proximal non-locking screw engaged with the first metatarsal bone; g) using the proximal non-locking screw as a pivot axis, manipulating the metatarsal fragments to obtain hallux valgus correction; h) inserting a distal non-locking screw through the distal non-threaded hole of the plate into the first metatarsal bone; i) tightening both non-locking screws to secure the straight section of the plate against the dorsal portion of the first metatarsal bone; j) inserting locking screws in the threaded holes of the proximal and distal legs; k) tightening both locking screws to secure the proximal and distal legs to the medial portion of the first metatarsal bone and complete final fixation across the osteotomy cut; and l) excising remaining medial bone caused by correction in step g) and closing the site.

Following tightening of both non-locking screws, the legs may be further bent to adjust to the particular shape of the fragmented metatarsal bone. The step of manipulating the metatarsal fragments includes pulling the plantar metatarsal fragment medially and the dorsal metatarsal fragment laterally. The non-locking screws are fixed perpendicularly to the osteotomy cut to achieve compression across the osteotomy. The non-locking screws may be angled up to 15° in the plate. Heads of the non-locking and locking screws lie flush with an upper surface of the plate upon the tightening of both the non-locking and locking screws.

### BRIEF DESCRIPTION OF THE DRAWING

The drawings illustrate the best mode presently completed under 35 USC §112.

FIG. 1 is a dorsal perspective view of an exemplary internal plate fixation device mounted on a metatarsal bone following a hallux valgus correction;

FIG. 2 is top plan view of one example of the internal plate fixation device shown in FIG. 1;

FIG. 3 is a front view of the example shown in FIG. 2;

FIG. 4 is a bottom view of the example shown in FIG. 2;

FIG. 5 is a right side view of the example shown in FIG. 2;

FIG. 6 is one perspective view of the example shown in FIG. 2 provided with non-locking and locking screws;

FIG. 7 is a perspective exploded view similar to FIG. 6;

FIG. 8 is another perspective view similar to FIG. 6;

FIG. 9 is a perspective view of a partial Ludloff osteotomy cut made in the first metatarsal bone to correct the hallux valgus deformity;

FIG. 10 is a perspective view of the initial positioning of the internal plate fixation device on the first metatarsal bone following the partial osteotomy cut;

FIG. 11 is a perspective view of the placement of a first non-locking screw in the internal fixation device;

FIG. 12 is a perspective view of the partial insertion of the first non-locking screw in the internal plate fixation device using a screwdriver;

FIG. 13a is a plan view of the internal plate fixation device temporarily fixed on the first metatarsal bone before hallux valgus correction;

FIG. 13b is a view similar to FIG. 13a showing the internal plate fixation device temporarily fixed on the first metatarsal bone following hallux valgus correction; and

FIG. 14 is a perspective view of the insertion of a second non-locking screw in the internal plate fixation device following completion of the osteotomy cut and hallux valgus correction.

### DETAILED DESCRIPTION

For purposes of promoting an understanding of the principles of the invention, reference will now be made to the examples illustrated in the drawings and described in the following written specification. It is understood that no limitation to the scope of the invention is thereby intended. It is further understood that the present invention includes any alterations and modifications to the illustrated example and includes further applications of the principles of the invention as would normally occur to one skilled in the art to which this invention pertains.

### Prior Hallux Valgus Correction and Fixation

A hallux valgus deformity of the forefoot is characterized by lateral deviation (i.e. away from the center line of the body) of the hallux formed by the proximal phalanx and the distal phalanx, and medial deviation (i.e. towards the center of the body, hallux varus) of the first metatarsal bone or metatarsus. This condition can lead to painful motion of the first metatarsal phalangeal (MTP) joint, and adjustments in gait that may ultimately cause problems further up the leg.

Historically, many different surgical procedures have been proposed to correct hallux valgus deformities. In the majority of cases, the surgical treatment involves an osteotomy to correct structural deformities associated with the first metatarsal bone and the hallux phalanges, relieve symptoms and pain and restore proper foot function. The osteotomy results in the creation and correction of metatarsal segments used to return the metatarsus and MTP joint to their normal anatomical positions and restore acceptable hallux valgus. In any osteotomy, it is essential that a fixation device, such as screws, K-wires, pins, plates and the like, be used to maintain correction of the metatarsal segments until bone union occurs.

One well known surgical procedure used for correcting hallux valgus deformities is the Ludloff osteotomy. The Ludloff osteotomy was first described in 1918 as a through and through oblique osteotomy in the transverse plane extending dorsal-proximal to plantar-distal when viewed in the sagittal plane (from the side). The Ludloff osteotomy allows for intermetatarsal angle reduction, lengthening and plantar displacement, but is not intrinsically stable, and therefore strong and reliable fixation is necessary. A Ludloff osteotomy surgical procedure entails making a dorsomedial skin incision extending from the first metatarsal phalangeal joint to the base of the first metatarsal joint. Once exposure is obtained, an osteotomy is performed which cuts the metatarsal bone into plantar and distal metatarsal fragments. The Ludloff osteotomy starts distal to the base of the first metatarsal, angled approximately 30°, extends from dorsal-proximal to plantar-distal, and ends just before the sesamoid complex at the first metatarsal distal base. About the dorsal two-thirds of the Ludloff osteotomy is performed starting at the dorsal-proximal site towards the plantar-distal site.

At the approximate one-third position of the osteotomy cut, the surgeon will temporarily fixate the metatarsal bone at the proximal-dorsal location using a first screw and/or a K-wire inserted without full compression in a proximal aspect of the dorsal metatarsal fragment. This temporary fixation, before the Ludloff osteotomy is completed, allows the surgeon to rotate the fragmented metatarsal bone after completing the Ludloff osteotomy cut. The Ludloff osteotomy cut is then completed and the metatarsal bone is gently shifted laterally using the temporary fixation point as an axis swivel to obtain hallux valgus correction of the metatarsal bone. This is performed by pulling the plantar metatarsal fragment medially and the dorsal metatarsal fragment laterally with manual pressure applied to the medial aspect of the first metatarsal cut. After the desired correction is confirmed fluoroscopically, the first screw is tightened to secure the osteotomy site at the proximal aspect, and a second screw, such as guided by a K-wire, is inserted across the distal aspect of the osteotomy site and tightened to complete fixation. Once approximate correction has been made, the medial eminence of the first metatarsal head is excised and the surgical site is closed.

While the conventional Ludloff osteotomy described above continues to be used, the previous fixation of the osteotomy with only two screws has been unsatisfactory in providing the rigid stabilization and precise fixation desired, and requires non-weight bearing post operative care delaying the normal walking gait cycle for the patient. The use of the known two screw fixation may lead to loss of correction between the metatarsal fragments and results in post operative weight bearing being transferred to the osteotomy. Further, the two screw fixation can result in undesirable tissue migration in the area of the osteotomy.

### The Present Fixation Device and Method of Use

Referring now to the drawings, the present disclosure describes a fixation device 10 and method of use for obtaining a more rigid stable and precise fixation of the hallux valgus correction in a Ludloff osteotomy than previously known.

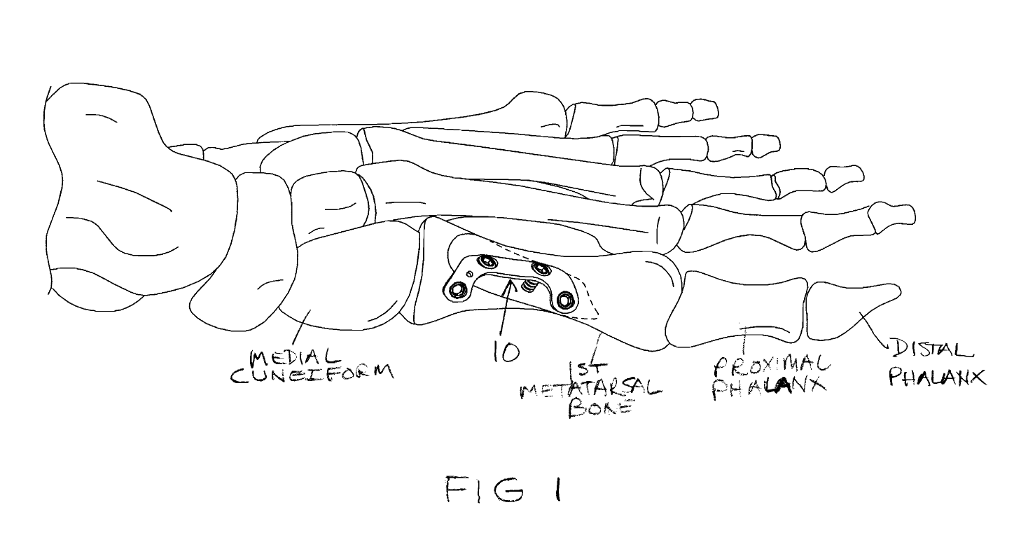

FIG. 1 illustrates a left forefoot including a medial cuneiform, a first metatarsal bone, a proximal phalanx and a distal phalanx. The fixation device 10 is shown mounted to the first metatarsal bone upon completion of a Ludloff osteotomy to correct the hallux valgus deformity.

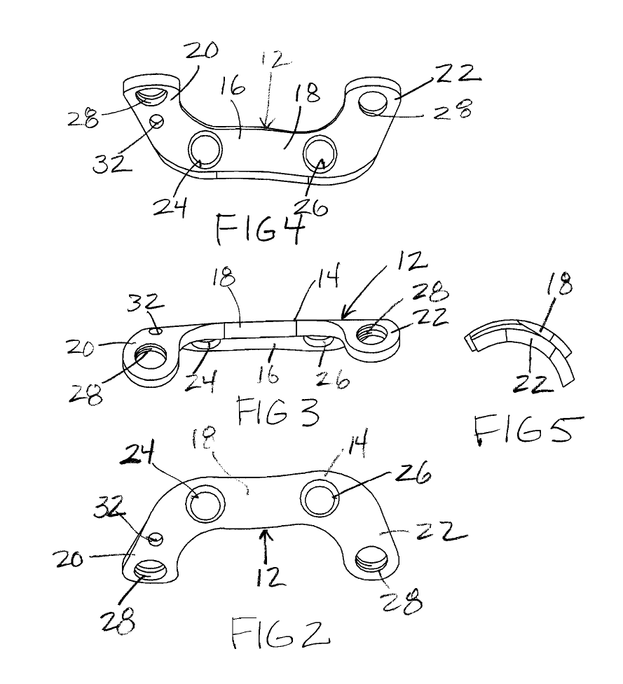

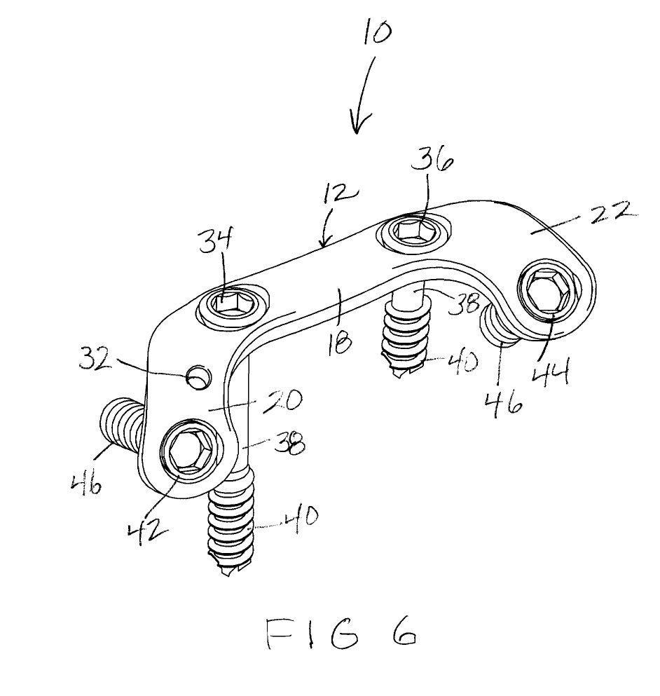

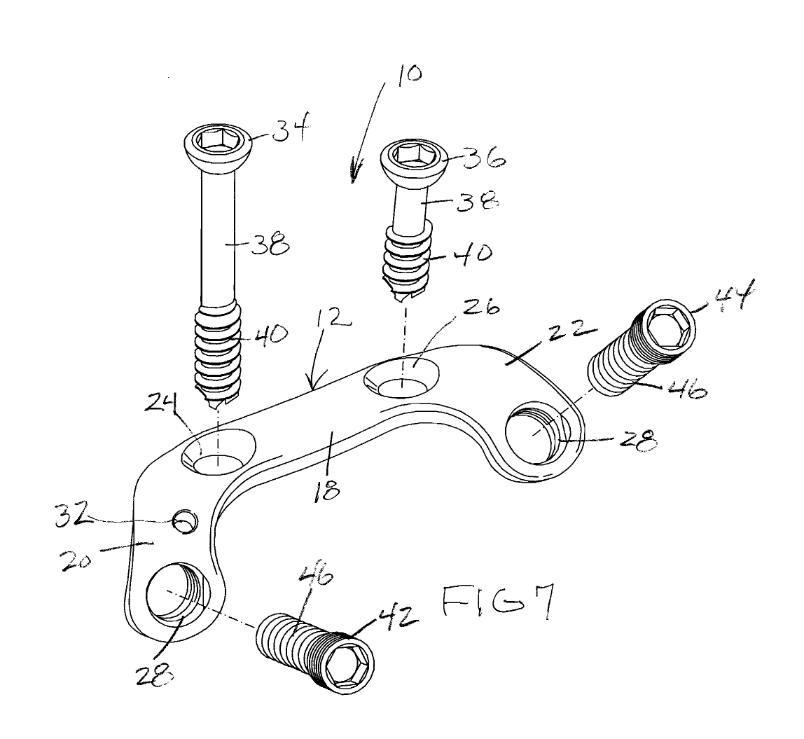

Referring to FIGS. 2-5, the fixation device 10 includes a unitary U-shaped internal fixation plate 12 having an upper surface 14 and a lower surface 16 configured with a curvature for substantially flush mounting on the outer curved surface of the first metatarsal bone. The plate 12 has a straight section 18 that transfers to and interconnects a pair of opposed proximal and distal legs 20, 22, respectively, which are bent rearwardly of the straight section 18 into an initial curvature. The proximal leg 20 is formed with a length that is slightly longer than the length of the distal leg 22. The straight section 18 is configured to engage a dorsal portion of the first metatarsal bone, and the legs 20, 22 are anatomically configured to engage a medial portion of the first metatarsal bone.

In the exemplary embodiment shown, the straight section 18 is configured with a thickness which is greater than the thickness of the legs 20, 22 for a purpose to be appreciated hereafter. Typically, the straight section 18 has a thickness of 1.8 millimeters and the bended legs have a thickness of 1.5 millimeters. The plate 12 can be constructed of titanium, stainless steel, plastic or other suitable material, and is preferably made of a titanium alloy Ti-6A1-4V. Each plate 12 has variable lengths of 31 millimeters, 34 millimeters, and 38 millimeters, for example, and is designed for use on the right or left forefoot. Each plate 12 has a slightly different curvature due to its length to accommodate for the particular anatomy and lateral correction. For example, a plate 12 of 31 millimeter length has a greater curvature than a plate 12 of 34 millimeter length which, in turn, has a curvature greater than a plate 12 of 38 millimeter length. The lower surface 16 and peripheral edges of plate 12 are rounded smooth surfaces to reduce irritation in use on the first metatarsal bone and surrounding tissue.

The plate 12 includes a plurality of set of holes that extend completely through the thickness of the plate 12. Such holes are non-locking (i.e. non-threaded) or locking (i.e. threaded) for receiving K-wires or bone fasteners in the form of cannulated (hollow) screws which are known to slide over the K-wires. More specifically, opposite ends of the straight section 18 are provided with a proximal non-threaded hole 24 and a distal non-threaded hole 26. Each leg 20, 22 is formed with a threaded hole 28 located at an outermost or distal end 30 of the leg. Diameters of non-threaded holes 24, 26 are substantially equal, while the diameters of the threaded holes 28 are differently sized. The non-threaded holes 24, 26 define temporary and final fixation holes which are used in the fixation of the first metatarsal bone. The threaded holes 28 define final fixation holes which are used in the fixation of the first metatarsal bone. A small hole 32 used for temporary fixation of the plate 12 is formed through the proximal leg 20 between the proximal non-threaded hole 24 in the straight section 18 and the threaded hole 28 in the proximal leg 20. The hole 32 has a diameter which is smaller than any of the non-threaded and threaded holes 24, 26, 28.

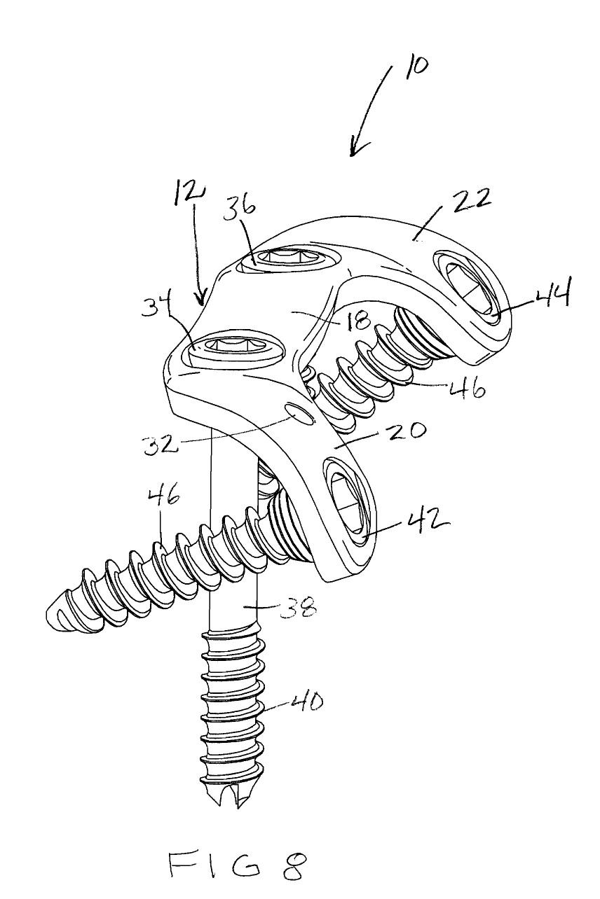

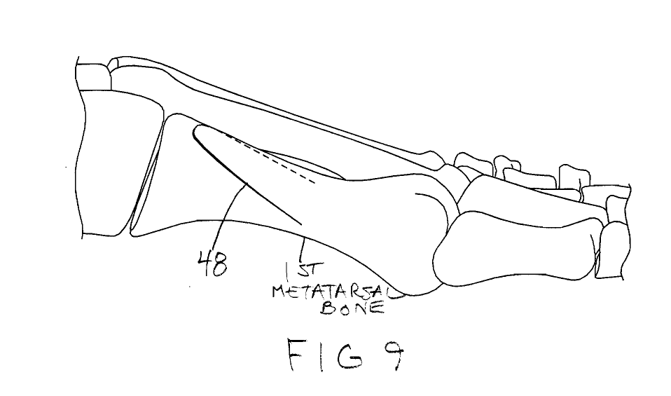

Referring to FIGS. 6-8, a proximal non-locking screw 34 is received in the proximal non-threaded hole 24, and a distal non-locking screw 36 is received in the distal non-threaded hole 26. A non-locking screw as used herein is any screw that is not locked to the plate by threads. Walls forming the non-threaded holes 24, 26 are appropriately configured to receive heads of the non-locking screws 34, 36 so that the non-locking screws may toggle to assume a plurality of angles relative to central axes of the non-threaded holes 24, 26. In the design shown, the non-locking screws 34, 36 are permitted to be multi-directional and can have concentric angulation up to 15° from the central axes of the non-threaded holes 24, 26. The non-locking screws 34, 36 have shafts 38 which are partially threaded at 40 along lower portions thereof. In the exemplary embodiment, the non-locking screws 34, 36 typically have equal diameters of 3 millimeters with proximal non-locking screw 34 typically having a length of 22-28 millimeters and the distal non-locking screw 36 typically having a length of 16-20 millimeters.

A proximal locking screw 42 is received in the threaded hole 28 of the proximal leg 20, and a distal locking screw 44 is received in the threaded hole 28 of the distal leg 22. A locking screw as used herein is any screw that is locked by threads to the plate 12. The locking screws 42, 44 have shafts 46 which are variously threaded along substantially entire lengths thereof. The proximal locking screw 42 typically has a 3.5 millimeter diameter and a length of 18-24 millimeters while the distal locking screw 44 typically has a 3 millimeter diameter and a length of 12-18 millimeters. The non-locking and locking screws 34, 36, 42, 44 are preferably formed of the same material as the plate 12.

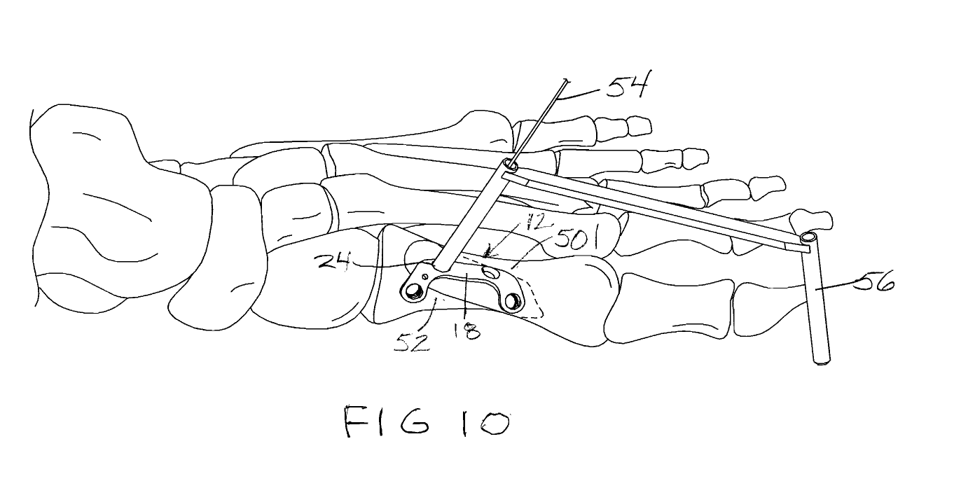

In use, the fixation device 10 is particularly useful during and after a Ludloff osteotomy of the first metatarsal bone and a hallux valgus correction. The Ludloff osteotomy commences with a medial based incision such that the dorsal medial cutaneous nerve is protected in the dorsal flap and exposure to the first metatarsal bone is achieved. A lateral release of the distal soft tissue is performed by surgeon preference. Using the metatarsal cuneiform joint as a marker, the Ludloff osteotomy cut is performed medially of the metatarsal bone and begins dorsally at the level of the metatarsal cuneiform joint. A three-quarter cut 48 extends obliquely at a 30° angle from dorsal-proximal to plantar-distal ending proximal to the sesamoid complex (FIG. 9).

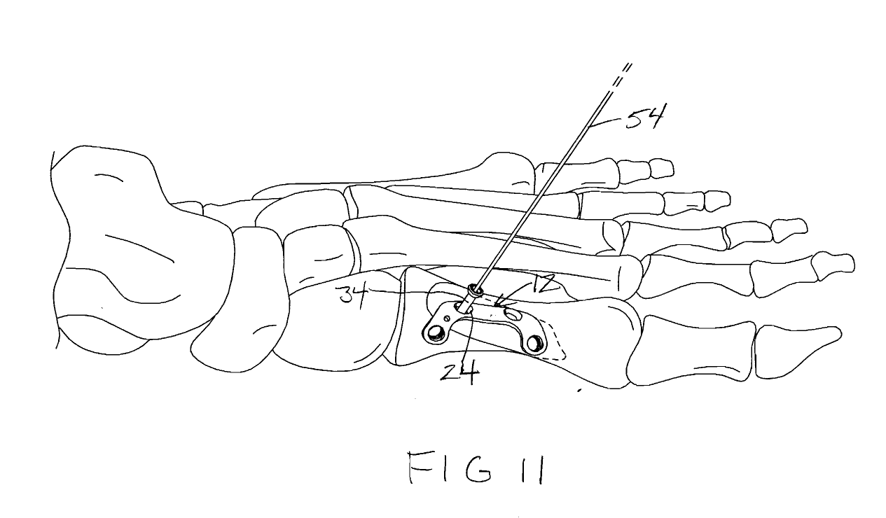

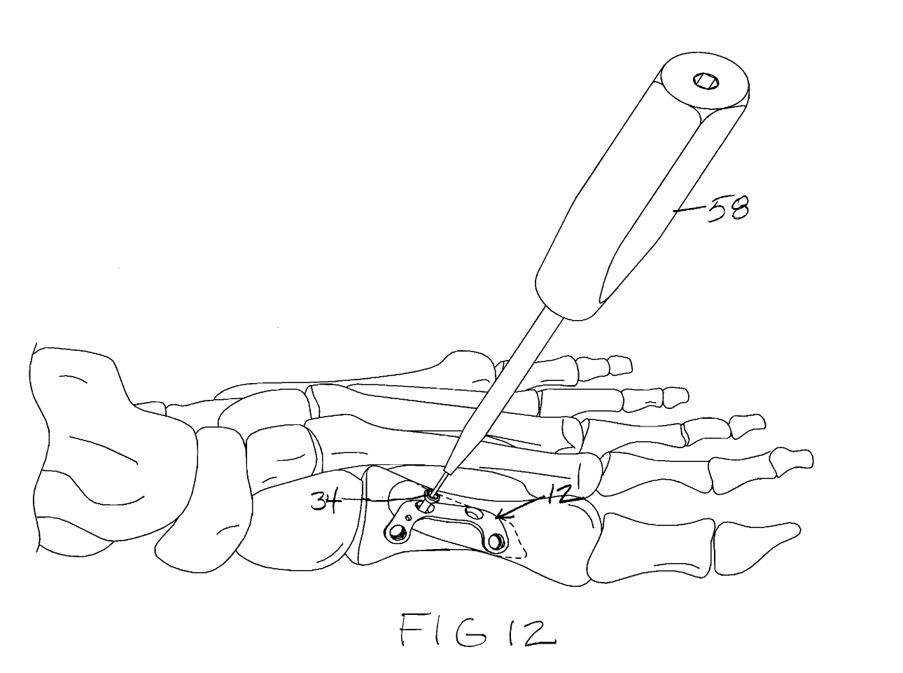

Referring to FIG. 10, once the three-quarter cut has been made, the fixation plate 12 is manually positioned across the partial osteotomy cut such that the straight section 18 engages the dorsal portion 50 of the first metatarsal bone, and the legs 20, 22 engage a medial portion 52 of the first metatarsal bone. Holding the plate manually or fixating the plate 12 with the temporary K-wire hole 32, a 150 millimeter length of K-wire 54 is inserted perpendicularly through the non-threaded hole 24 using, for example, a K-wire drill guide 56. The K-wire 54 is typically used with a depth gauge to measure an appropriate depth for the proximal non-locking screw 34 which is inserted over the K-wire 54 through the non-threaded hole 24 and placed across the osteotomy as shown in FIG. 11. The non-locking screw 34 is then partially inserted using a screwdriver 58 as depicted in FIG. 12. The placement of the proximal non-locking screw 34 enables a temporary fixation and defines a pivot axis used in the hallux valgus correction.

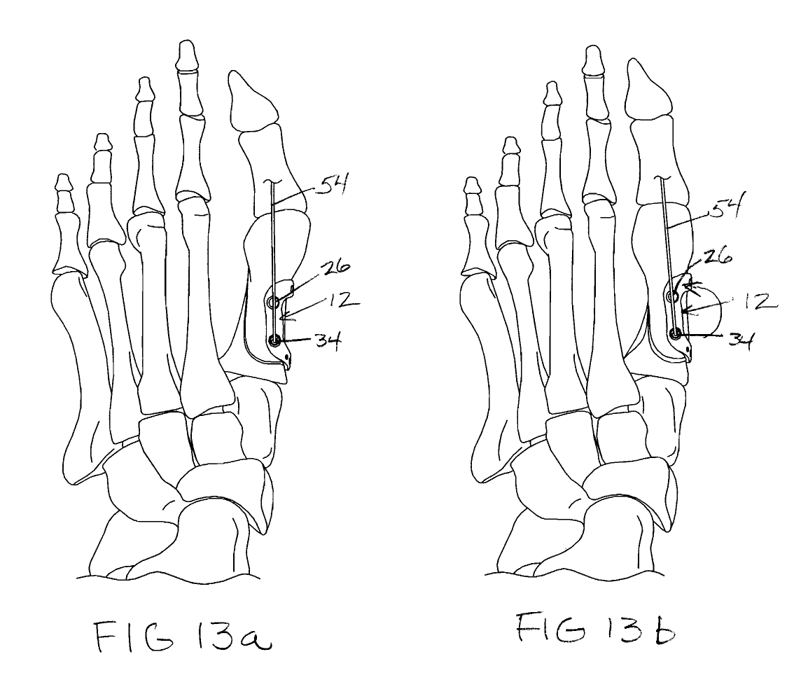

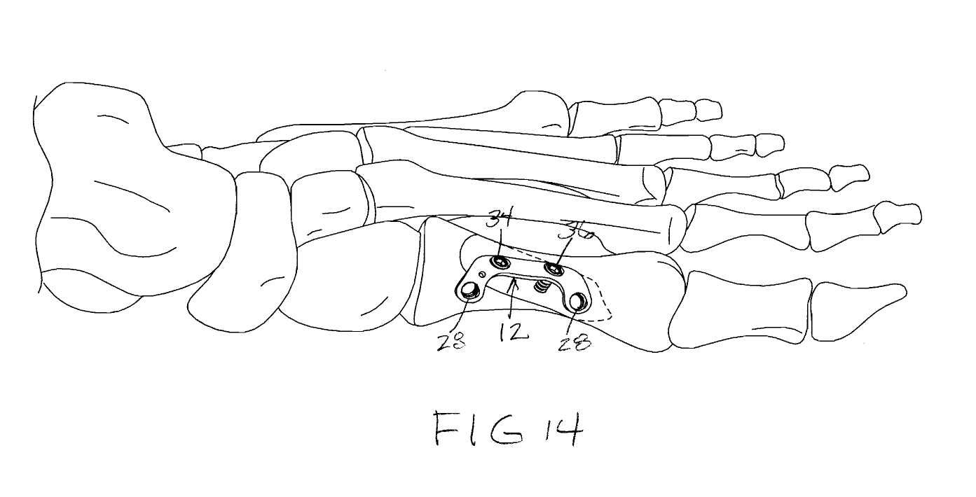

The remainder of the osteotomy cut is completed to create dorsal and plantar metatarsal fragments with the plate 12 and the non-locking screw 34 in position on the metatarsal bone. At this point, using the non-locking screw 34 as the pivot axis, the surgeon performs a hallux valgus correction by pulling the plantar metatarsal fragment medially, and the dorsal metatarsal fragment laterally with manual pressure applied to the medial head of the first metatarsal bone. FIG. 13a shows the uncorrected position of the first metatarsal bone and FIG. 13b shows the corrected position of the first metatarsal bone. The surgeon may wish to use a towel clamp or the like to hold the corrected position so that a K-wire can next be inserted into the distal non-threaded hole 26 for guiding the distal non-locking screw 36 thereto. Once a proper depth has been determined for the screw 36, both the proximal and distal non-locking screws 34, 36 are tightened in the bone (FIG. 14) so that they extend perpendicularly to the osteotomy with the heads of the screws 34, 36 lying flush with the upper surface of the straight section of the plate 12. It should understood that during the placement of the proximal and distal non-locking screws 34, 36, the respective receiving holes 24, 26 will allow concentric angulation so that the screws 34, 36 achieve their proper perpendicular orientation. After tightening of the non-locking screws 34, 36, locking screws 42, 44 are inserted in the threaded holes 28 of the legs 20, 22 and tightened to the bone to complete final fixation of the osteotomy with the heads of the locking screws 42, 44 lying flush with the upper surface of the legs 20, 22 as shown in FIG. 1. A surgeon then excises any remaining medial bone caused by the hallux valgus correction and closes the osteotomy site.

If desired, the legs 20, 22 may be further bent to adjust to the particular shape of the fragmented first metatarsal bone before final fixation. This is made possible due to the lesser thickness in the legs 20, 22 as compared to the straight section 18.

It is contemplated that the internal fixation plate 12, described herein, can be provided in a kit or case with plates 12 of multiple sizes for the right and left forefoot with multiple locking and non-locking screws 34, 36, 42, 44 and with various tools, instruments, guides and the like to give a surgeon flexibility in selecting the desired means for securing the plate 12 to the first metatarsal bone.

The fixation device shown and described provides non-load bearing fixation of the metatarsal correction for the Ludloff osteotomy. However, it should be appreciated that, in contrast with the prior art, the installed fixation device 10 is designed to withstand the weight bearing capacity of the normal human being and the weight bearing load displaced to the corrected human metatarsal bone when undergoing the normal walking gait cycle. Thus, while the fixation device allows for non-weight bearing union of the metatarsal bone, it provides the option of weight bearing on the metatarsal bone immediately after fixation of the plate 12 is applied to the Ludloff osteotomy. The non-locking dorsal-to-plantar screws 34, 36 fixed perpendicular to the osteotomy achieve compression across the osteotomy with rigid precise stabilization after osteotomy correction transferring the weight bearing to the plate 12 and not the osteotomy. In addition, the locking screws 42, 44 are inserted from the medial side of the first metatarsal bone after correction to gain stable locking fixation of the osteotomy so that the weight bearing is transferred to the plate 12. The plate 12 is slightly thicker dorsally across the straight section to provide strength across the osteotomy site, and is thinner in the legs 20, 22 to allow for bending of the plate 12 and permit low profile fixation of the plate 12 to the bone.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims, if they have structural elements that do not differ from the literal language of the claims, or if they include equivalent structural elements with insubstantial differences from the literal language of the claims.

Various alternatives and embodiments are contemplated as being within the scope of the following claims, particularly pointing out and distinctly claiming the subject matter regarded as the invention.

What is claimed is:

1. An internal fixation device for use in fixation of a metatarsal bone, the fixation device comprising:
a U-shaped plate having a straight section interconnecting a pair of opposed legs which are bent relative to the straight section,
wherein the straight section is formed therethrough with a set of non-threaded holes, and
wherein the legs are formed therethrough with a set of threaded holes.

2. The internal fixation device of claim 1, wherein the straight section includes a pair of spaced apart non-threaded holes.

3. The internal fixation device of claim 1, wherein each leg has a single threaded hole.

4. The internal fixation device of claim 1, wherein the non-threaded holes are located at opposite ends of the straight section.

5. The internal fixation device of claim 1, wherein the threaded holes are located adjacent outermost edges of the legs.

6. The internal fixation device of claim 1, wherein, in a top view of the plate, the legs are bent rearwardly of the straight section.

7. The internal fixation device of claim 1, wherein one of the legs has a length which is longer than the length of the other leg.

8. The internal fixation device of claim 1, wherein the straight section has a thickness that is greater than the thickness of the legs to allow bending of the legs beyond an initial bent curvature thereof.

9. The internal fixation device of claim 1, wherein the non-threaded holes define temporary and final fixation holes adapted to be used in fixation of the metatarsal bone.

10. The internal fixation device of claim 1, wherein the threaded holes define final fixation holes adapted to be used in fixation of the metatarsal bone.

11. The internal fixation device of claim 1, wherein the non-threaded holes are configured to receive non-locking screws therein.

12. The internal fixation device of claim 1, wherein the threaded holes are configured to receive locking screws therein.

13. The internal fixation device of claim 1, wherein the threaded holes are differently sized to receive differently sized locking screws.

14. The internal fixation device of claim 1, wherein one of the legs is formed with a temporary fixation hole adapted to be used in fixation of the metatarsal bone.

15. The internal fixation device of claim 1, wherein the plate is configured to extend across an osteotomy of the metatarsal bone.

16. The internal fixation device of claim 1, wherein the straight section is configured to engage a dorsal portion of the metatarsal bone.

17. The internal fixation device of claim 1, wherein the legs are configured to engage a medial portion of the metatarsal bone.

18. The internal fixation device of claim 1, wherein the plate is configured to be engaged on the metatarsal bone during and after a Ludloff osteotomy.

19. An internal fixation device for use in fixation of a metatarsal bone, the fixation device comprising:
a U-shaped plate having a straight section interconnecting a pair of proximal and distal opposed legs which are bent relative to the straight section, wherein the straight section is formed therethrough with a proximal non-threaded hole and a distal non-threaded hole, and wherein each leg is formed therethrough with a threaded hole at a distal end of each leg;
a proximal non-locking bone fastener received in the proximal non-threaded hole;
a distal non-locking bone fastener secured in the distal non-threaded hole;
a proximal locking bone fastener received in the threaded hole of the proximal leg; and
a distal locking bone fastener received in the threaded hole of the distal leg.

20. The internal fixation device of claim 19, wherein each bone fastener is a cannulated screw adapted to receive a K-wire.

21. The internal fixation device of claim 19, wherein the non-locking bone fasteners includes shafts which are partially threaded along lower portions thereof.

22. The internal fixation device of claim 19, wherein the locking bone fasteners include shafts which are threaded along substantially entire lengths thereof.

23. The internal fixation device of claim 19, wherein the walls forming non-threaded holes are configured such that the bone fasteners received therein toggle to assume a plurality of angles relative to central axes of the non-threaded holes.

24. The internal fixation device of claim 19, wherein the straight section and the non-locking bone fasteners are configured to engage a dorsal portion of the metatarsal bone.

25. The internal fixation device of claim 19, wherein the legs and locking bone fasteners are configured to engage a medial portion of the metatarsal bone.

26. The internal fixation device of claim 19, wherein the plate and bone fasteners are constructed of titanium alloy Ti-6A1-4V.

27. A surgical method of fixating a Ludloff osteotomy of a first metatarsal bone to correct a hallux valgus deformity utilizing the internal fixation device of claim 1.

28. A surgical method of fixating a Ludloff osteotomy of a first metatarsal bone to correct a hallux valgus deformity, the method comprising the steps of:
(a) providing a U-shaped plate configured to be engaged on a first metatarsal bone across the Ludloff osteotomy, the plate having a straight section interconnecting a pair of proximal and distal opposed legs which are bent relative to the straight section, wherein the straight section is formed therethrough with a proximal non-threaded hole and a distal non-threaded hole, and wherein each leg is formed therethrough with a threaded hole at a distal end of each leg;
(b) entering the tissues of the foot affected with the hallux valgus deformity using a medial incision at a site on the first metatarsal bone to gain exposure thereto;
(c) from the medial site of the first metatarsal bone, performing a partial three-quarter Ludloff osteotomy at the site extending obliquely from dorsal-proximal to plantar-distal;
(d) positioning the plate across the partially osteotomy cut such that the straight section engages a dorsal portion of the first metatarsal bone, and the legs engage a medial portion of the first metatarsal bone;
(e) temporarily fixating the plate at a proximal-dorsal location of the partial cut by inserting a proximal non-locking screw through the proximal non-threaded hole of the plate into the first metatarsal bone;
(f) completing the osteotomy cut forming dorsal and plantar metatarsal fragments with the plate and proximal non-locking screw engaged with the first metatarsal bone;
(g) using the proximal non-locking screw as a pivot axis, manipulating the metatarsal fragments to obtain hallux valgus correction;
(h) inserting a distal non-locking screw through the distal non-threaded hole of the plate into the first metatarsal bone;
(i) tightening the proximal and distal non-locking screws to secure the straight section of the plate against the dorsal portion of the first metatarsal bone;
(j) inserting locking screws in the threaded holes of the proximal and distal legs;
(k) tightening both locking screws to secure the proximal and distal legs to the medial portion of the first metatarsal bone and complete fixation across the osteotomy cut; and
(l) excising remaining medial bone caused by correction in step (g) and closing the site.

29. The method of claim 28, wherein, following tightening of both non-locking screws, the legs are further bent to adjust to the particular shape of the fragmented metatarsal bone.

30. The method of claim 28, wherein the step of manipulating the metatarsal fragments includes pulling the plantar metatarsal fragment medially and the dorsal metatarsal fragment laterally.

31. The method of claim 28, wherein the non-locking screws are fixed perpendicularly to the osteotomy cut to achieve compression with the plate across the osteotomy.

32. The method of claim 28, wherein the non-locking screws are angled up to 15° in the plate.

33. The method of claim 28, wherein the heads of the non-locking and locking screws lie flush with the plate upon tightening of both the locking and non-locking screws.
